(19) United States
(12) Patent Application Publication
(10) Pub. No.: US 2011/0207074 A1
(43) Pub. Date: Aug. 25, 2011

(54) DENTAL IMAGING SYSTEM AND METHOD

(76) Inventors: Olaf Andrew Hall-Holt, Northfield, MN (US); Bruce Willard Hultgren, Victoria, MN (US)

(21) Appl. No.: 12/912,636

(22) Filed: Oct. 26, 2010

Related U.S. Application Data
(60) Provisional application No. 61/254,904, filed on Oct. 26, 2009.

(51) Int. Cl. A61C 19/04; A61C 19/02; A61B 6/14

(52) U.S. Cl. 433/29; 433/215; 206/63.5

(57) ABSTRACT

A dental slurry is used with a digital camera to create a three dimensional model of target teeth. The dental slurry includes granular particles suspended in a pharmacologically acceptable carrier. The slurry is used to cover target teeth with a large number of particles. Overlapping digital images are taken of the target teeth and provided to a computer. An algorithm is used to find a correspondence for each feature in the overlapping digital images by error analysis. Once enough correspondences are found, the algorithm creates a three dimensional electronic model from the plurality of two dimensional images based on a best fit error analysis.

### RELATED PATENT APPLICATION & INCORPORATION BY REFERENCE

This application claims priority to U.S. Provisional Patent Application Ser. No. 61/254,904, filed on Oct. 26, 2009 in the United States and which application is incorporated herein by reference. A claim of priority, to the extent appropriate is made.

### FIELD OF THE INVENTION

The present invention relates generally to an imaging system. More particularly, the invention relates to creating three dimensional images of portions of a human body from overlapping two dimensional images that include a plurality of markers on the portion of the human body. More particularly still, an imaging system, kit and method of the present invention uses a dental slurry and an intra-oral digital camera to record overlapping two dimensional images of target teeth to create a three dimensional model of the target teeth and surrounding dentition.

### BACKGROUND

Generally preparing orthodontic treatment plans and fabricating dental prostheses includes creating an impression of the target teeth. As is well known in the art, an impression is made by biting into a material held in an impression tray. Thus, the impression captures a negative three dimensional image of the teeth. The impression material can then be used to create a positive image plaster study cast. Although providing accurate positive images of the teeth of interest, there are several drawbacks associated with this technique. First, the process requires a substantial amount of time to take the impression and then create the study cast. Second, the material in the impression tray, and the impression tray itself, may be uncomfortable or unpleasant tasting to the patient. Third, the necessary storage and identification of study casts can be costly.

To overcome the drawbacks described above, several systems have been designed to create electronic three dimensional models of teeth. These systems include touch probe scanners, laser scanners and structured light systems. However, each of these devices has drawbacks including expense, complexity, and time.

Therefore, there is a need in the art for a system and method for creating an accurate electronic three dimensional model of target teeth and surrounding dentition. The present invention overcomes the shortcomings of the prior art and addresses these needs in the art.

### SUMMARY

The present invention includes a dental slurry (or colloid) having marker granules suspended therein. The slurry may be applied to target teeth by the patient swishing the slurry (e.g., in a manner associated with mouthwashes) or may be applied with an applicator brush, sponge or other applicator. The granules may be of varying shapes and sizes that temporarily are located and visible on the target teeth. The granules may be located within the slurry that sticks to the teeth and/or may stick or adhere directly to the target teeth and surrounding dentition. A digital camera is used to capture a plurality of two dimensional overlapping images of the target teeth. Unstructured light may be employed while capturing the two dimensional images. The resulting images of the granule covered target teeth are analyzed using an algorithm that selects common granules or markers between the images (e.g., common pixels or groups of pixels). The analysis of the common granules or markers results in the identification of a correspondence for each granule or marker. The combination of the calculated correspondence and the calculated or known position for the camera used to take each image provides the algorithm with the necessary data to create a three dimensional electronic model of the target teeth. Once the three dimensional model of the target teeth is created, it may be subsequently stored, modified, used by the doctor and/or displayed on a video display unit.

The present invention further includes the application of a composition having markers suspended therein, to a target area. The composition may be applied to the target area by brushing, spraying, painting, rubbing, or by use of another applicator. The markers may be of varying shapes and sizes that temporarily are located and visible on the target surface. The markers may be located within the composition that sticks to the target area and/or may stick or adhere directly to the target area. A digital camera is used to capture a plurality of two dimensional overlapping images of the target area. Unstructured light may be employed while capturing the two dimensional images. The resulting images of the marker covered target area are analyzed using an algorithm that selects common markers between the images (e.g., common pixels or groups of pixels). The analysis of the common markers results in the identification of a correspondence for each marker. The combination of the calculated correspondence and the calculated or known position for the camera used to take each image provides the algorithm with the necessary data to create a three dimensional electronic model of the target area. Once the three dimensional model of the target area is created, it may be subsequently stored, modified, used by an operator and/or displayed on a video display unit.

In one embodiment constructed in accordance with the principles of the present invention, a digital intra-oral camera is employed. The digital intra-oral camera may include multiple cameras set in a fixed relationship and aspect ratio. Each camera may include a protected lens at the end of a wand structure to direct the light to the appropriate CCD (or CMOS) camera devices. Thus, a single wand structure may hold multiple cameras in a fixed relationship and aspect ratio. The multiple camera arrangement allows the user to simultaneously capture several images of the same surface. One advantage of a multiple-camera embodiment is that, generally, the accuracy of the three dimensional rendering of the target teeth increases as the number of images of the target teeth taken from different known positions increases. Another advantage of this type of camera is that it eliminates the use of impression trays and materials. Instead, a relatively small intra-oral camera may be rapidly inserted and removed from the intra-oral cavity to meet the patient's needs as the images are captured.

Therefore, according to one aspect of the invention, there is provided a dental slurry for creating an electronic three dimensional model of target teeth using unstructured light, comprising: at least one inert or pharmacologically acceptable carrier; and granular particles suspended in the carrier. According to another aspect of the invention, there is provided, a method for creating electronic three dimensional images of target teeth using unstructured light, comprising: swishing a dental slurry having particles suspended therein within the mouth of a patient; acquiring overlapping images of target teeth covered by the particles; combining the images into a three dimensional electronic model using an algorithm that finds correspondences for distinct particles based on error analysis; and displaying the three dimensional electronic model. According to a third aspect of the invention, there is provided a kit for producing three dimensional images of target teeth, comprising: a container, wherein the container holds granular particles and an inert or pharmacologically acceptable carrier constituting a dental slurry. According to a fourth aspect of the invention, a system is provided for producing three dimensional images of target teeth comprising: a dental slurry, wherein the dental slurry comprises granular particles and inert or pharmacologically acceptable agent; a digital camera; a source of unstructured light; a computing system; and a display.

According to yet another aspect of the invention, there is provided a method for creating electronic three dimensional images of facio-cranial targets using unstructured light comprising: applying a slurry having particles suspended therein onto the facio-cranial target; acquiring overlapping images of the facio-cranial target covered by the particles; combining the images into a three dimensional electronic model using an algorithm that finds correspondences for distinct particles based on error analysis; and displaying the three dimensional electronic model. Further according to this aspect of the invention, the facio-cranial target may be a dental device, such as a study cast and the slurry may be applied to the study cast by spraying or dipping. The facio-cranial target might also be a dental prosthesis, a dental implant, a dental appliance or a bite registration wafer.

According to yet another aspect of the invention, there is provided a method for creating electronic three dimensional images of target areas using unstructured light comprising: applying a composition having markets suspended therein onto the target area; acquiring overlapping images of the target area covered by the markers; combining the images into a three dimensional electronic model using an algorithm that finds correspondences for distinct markers based on error analysis; and displaying the three dimensional electronic model. Further according to this aspect of the invention, the target area may be a medical device, such as a cast and the composition may be applied to the cast by spraying or dipping. The target area might also be a prosthesis, an implant, a dental appliance or a bite registration wafer.

While the invention will be described with respect to preferred embodiment configurations and with respect to particular devices used therein, it will be understood that the invention is not to be construed as limited in any manner by either such configuration or components described herein. Also, while particular slurries, cameras and computers are described herein, it will be understood that such particular colloids and devices are not to be construed in a limiting manner. Instead, the principles of this invention extend to any manner of applying contrasting or uniquely identifiable markers to facio-cranial targets of interest, capturing overlapping two dimensional images of the targets of interest, and using the uniquely identifiable markers common to a plurality of two dimensional images to create a three dimensional model. These and other variations of the invention will become apparent to those skilled in the art upon a more detailed description of the invention.

The advantages and features which characterize the invention are pointed out with particularity in the claims annexed hereto and forming a part hereof. For a better understanding of the invention, however, reference should be had to the drawings which form a part hereof and to the accompanying descriptive matter, in which there is illustrated and described a preferred embodiment of the invention.

The reference to granules or markers in the current invention is one example of a feature that may be used. It will be appreciated that when processing the images of the present invention that many features may be considered, including granules or markers. Accordingly, reference to granules or markers herein shall not be construed as limiting—e.g., since a feature may comprise naturally occurring markers on the target area, artificial markers on the target area, granules, particles, markers, darker areas of the target area, lighter areas of the target area, or any other natural or artificial object or data capable of providing pixel data to form correspondences or for other types of image analysis.

While the embodiment(s) of the invention is described in an intra-oral environment and uses an intra-oral imaging system and method for calculating the three dimensional rendering of target teeth, one of skill in the art will appreciate that the present invention may be used in other environments. For example, rather than target teeth, a three dimensional model of other facio-cranial features may be desired. Further, other reconstructive surgery sites, other human appendages, and inanimate objects may be of interest. In some cases, rather than a granular slurry, appropriate geometric patterns/dots may be placed or projected on the feature of interest. Thus, one of skill in the art will appreciate that the present invention transfers to other environments and applications.

### BRIEF DESCRIPTION OF DRAWINGS

FIG. 1 is a plurality of target teeth after swishing with a dental slurry. The target teeth are covered with granular particle markers.

FIG. 2 is a general schematic of steps in an embodiment of the present invention.

FIG. 3 is an embodiment of a system which implements the principles of the present invention.

FIG. 4 is an image of target teeth after application of a composition comprising carbon black and titanium dioxide.

FIG. 5 is a general schematic of steps in an embodiment of the present invention.

FIG. 6 is a mesh reconstruction from working example 2 of side view of a target tooth

FIG. 7 is a partial mesh reconstruction from working example 2 of a side view of a target tooth.

FIG. 8 is two overlapping images (A and B) of the surface of a target tooth used in working example 2.

### DETAILED DESCRIPTION

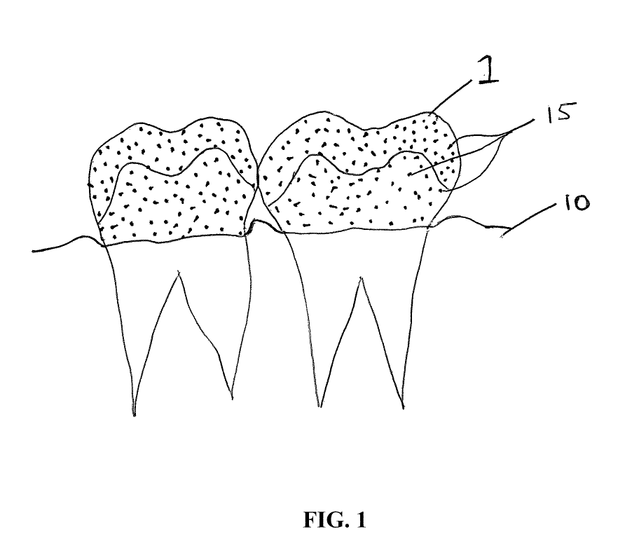

Referring now to the drawings wherein like reference numerals designate identical or corresponding parts throughout the several views, an embodiment of dental slurry is shown in FIG. 1. Target teeth 1 are rooted in dentition 10 and covered with granular particles 15 that are suspended within the slurry. The granular particles may be applied by having the patient swish the dental slurry around their mouth, after which the granular particles 15 temporarily cover the target teeth. In another embodiment the dental slurry is applied using an applicator brush or sponge to apply the markers to the target teeth. Some embodiments will employ a transparent or translucent mouthpiece, or similar apparatus, instead or in combination with a dental slurry. The transparent or translucent mouthpiece will have granular particles suspended within or affixed to the mouthpiece.

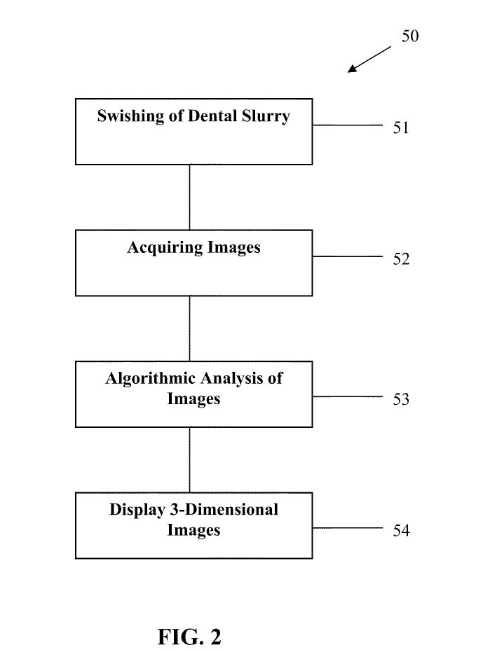

Turning now to FIG. 2 a logical flow of the steps included in a preferred embodiment method of the present invention is illustrated at 50. First, at step 51 the patient swishes the dental slurry around in their mouth to disperse the contrasting markers or uniquely identifiable granular particles on the patient's teeth. After swishing the dental slurry, a plurality of digital images are taken at step 52 of the target teeth from overlapping positions by a camera operatively attached to a single wand structure and utilizing unstructured light. In one embodiment, the single wand structure may include multiple cameras having a fixed relationship, fixed pixel width, and fixed aspect ratio. While a series of predetermined or preset locations may be used when capturing the images, such predetermined images are not required. The overlapping images are processed by a general purpose computer (e.g., a personal computer with a Pentium chipset manufactured by Intel) using an algorithm at step 53 that finds corresponding areas of the target teeth in different images. The corresponding areas are determined by analyzing multiple images of the same surface to determine the correspondence for a single granule particle or marker based on error analysis. Once the correspondences for a series of single markers on the target teeth are found, a three dimensional image of the target teeth is created and may be displayed at step 54.

It will be appreciated that the granules of the dental slurry temporarily bind (either directly or as part of the slurry) to the target teeth and act as a unique or distinguishable marker. It will be further appreciated that granules may be embedded within or affixed to an apparatus that covers or lies over the target teeth. In a preferred embodiment, the apparatus is a translucent or transparent mouthpiece that covers the target teeth, instead or in combination with a dental slurry. Upon insertion of the mouthpiece into a patient's mouth, the granules are visible in combination with target teeth, thereby permitting visualization of the underlying target teeth in combination with the overlying granules. In additional embodiments, the apparatus may be opaque with affixed granules. In alternative embodiments, the apparatus will be a film capable of adhering to the target teeth tightly enough to portray the features of the underlying target teeth. The film may be translucent, transparent or opaque. These unique or distinguishable markers are captured in the two dimensional images by the camera. The algorithm of the present invention includes a series of error based calculations using several parameters, that may include the fixed relationship of cameras, fixed pixels, fixed aspect ratio of overlapping images, the optical center of images, and the correspondence for each single marker to create the three dimensional model of the target teeth.

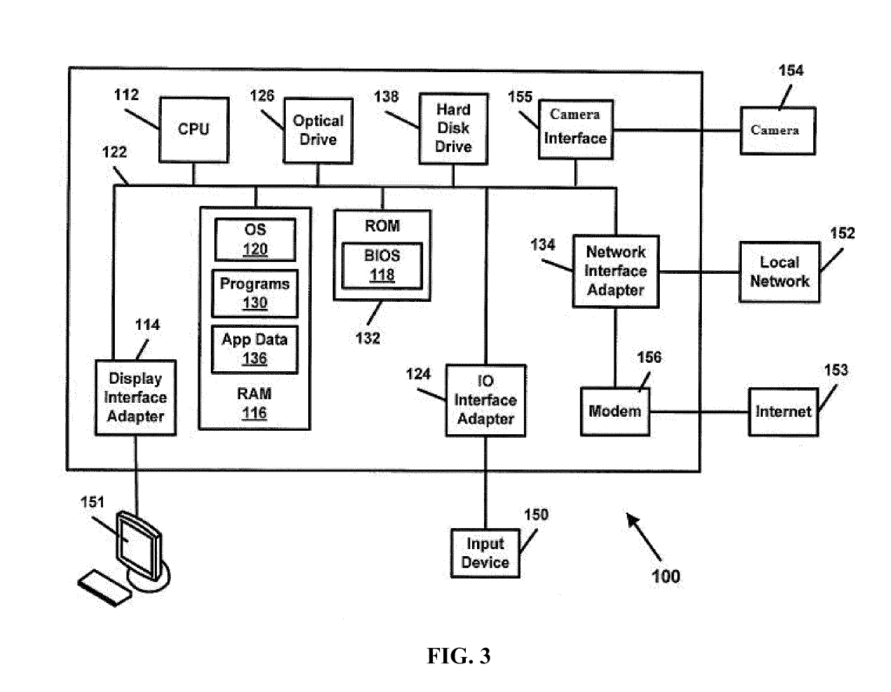

As noted above, a general-purpose computing system 100 may be employed with the present invention. Such a system 100 is shown in FIG. 3 and may be used to store and implement the algorithms, as well as provide guidance to the user and display resulting three dimensional images. It will be appreciated, however, that other types of computing systems may be used. The computer 100 includes a processor unit 112, read only memory (ROM) 132, random access memory (RAM) 116, and a system bus 122 that couples various system components including the RAM 116 to the processor unit 112. The system bus 122 may be any of several types of bus structures including a memory bus or memory controller, a peripheral bus and a local bus using any of a variety of bus architectures. A basic input/output system 118 (BIOS) contains basic routines that help transfer information between elements within the personal computer 100 and may be stored in ROM 132.

The personal computer 100 further includes a hard disk drive 138 for reading from and writing to a hard disk (not shown), a magnetic disk drive (not shown) for reading from or writing to a removable magnetic disk, and an optical disk drive 126 for reading from or writing to a removable optical disk such as a CD ROM, DVD, or other optical media. The hard disk drive 138, magnetic disk drive, and optical disk drive 126 are connected to the system bus 122 by a hard disk drive interface (not shown), a magnetic disk drive interface (not shown), and an optical drive interface (not shown), respectively. The drives and their associated computer-readable media provide nonvolatile storage of computer readable instructions, data structures, programs, and other data for the personal computer 100.

Although the exemplary environment described herein employs a hard disk drive 138, a removable magnetic disk, and removable optical disk drive 126, other types of computer-readable media capable of storing data may be used in the exemplary system. Examples of these other types of computer-readable mediums that may be used in the exemplary operating environment include magnetic cassettes, flash memory cards, digital video disks, Bernoulli cartridges, random access memories (RAMs), and read only memories (ROMs).

A number of program modules may be stored on the hard disk drive 138, magnetic disk drive, optical disk drive 126, ROM or RAM, including an operating system 120, one or more application programs 130, other program modules (not shown), and program (i.e., application) data 136. A user may enter commands and information into the personal computer 100 through input devices such as a keyboard and/or mouse 150 (or other pointing device). Examples of other input devices may include a microphone, joystick, game pad, satellite dish, and camera 154. These and other input devices are often connected to the processing unit 112 through an I/O port interface 124 coupled to the system bus 122. Nevertheless, these input devices also may be connected by other interfaces, such as a parallel port, game port, or a universal serial bus (USB). A monitor 151 or other type of display device is also connected to the system bus 122 via an interface, such as a video adapter 114. In addition to the monitor, personal computers typically include other peripheral output devices (not shown), such as speakers and printers.

The personal computer 100 may operate in a networked environment using logical connections to one or more remote computers. The remote computer may be another personal computer, a server, a router, a network PC, a peer device or other common network node, and typically includes many or all of the elements described above relative to the personal computer 100. The network connections include a local area network (LAN) and a wide area network (WAN). Such networking environments are commonplace in offices, enterprise-wide computer networks, intranets, and the Internet.

When used in a LAN networking environment, the personal computer 100 is connected to the local network 152 through a network interface or adapter 110. When used in a WAN networking environment, the personal computer 100 typically includes a modem or other means for establishing communications over the wide area network, such as the Internet 153. The modem 156, which may be internal or external, is connected to the system bus 122 via the network interface adapter 134. In a networked environment, program modules depicted relative to the personal computer 100, or portions thereof, may be stored in the remote memory storage device. It will be appreciated that the network connections shown are exemplary only and other means of establishing a communications link between the computers may be used.

A camera 154 can be connected to the computer 100 via an appropriate camera interface 155. The camera interface 155 can be connected to the bus 122 such that the collected digital data may be stored in the appropriate or desired memory location, manipulated by the CPU 112, displayed on the display 151, etc. Cameras include CCD and CMOS cameras, as well as other cameras that capture digital information using unstructured light and which can be arranged and configured for collecting images of target teeth. The camera 154 may also connected to the computer 100 via a USB port to download the images, a memory disk/stick may be removed from the camera 154 and read by an appropriate reader connected the computer or the camera 154 may employ wireless communication (e.g., Bluetooth or other communication scheme) to transmit the captured two dimensional images to the computer 100.

Portions of the preferred embodiment constructed in accordance with the principles of the present invention utilize a computer and are described herein as implemented by logical operations performed by a computer. The logical operations of these various computer implemented processes are generally performed either (1) as a sequence of computer implemented steps or program modules running on a computing system and/or (2) as interconnected machine modules or hardware logic within the computing system. The implementation is a matter of choice dependent on the performance requirements of the computing system implementing the invention. Accordingly, the logical operations making up the embodiments of the invention described herein can be variously referred to as operations, steps, or modules. Further, although the computer may be described as proceeding from step to step, it will be appreciated that the computer is actually logically operating a series of instructions and commands.

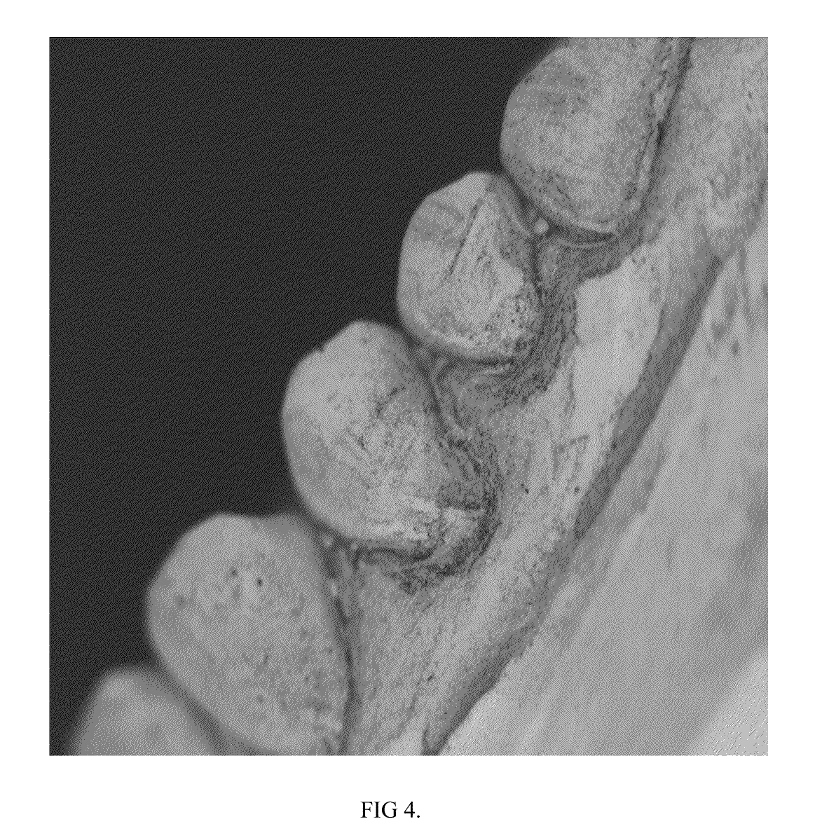

Turning now to FIG. 4 an image of target teeth captured using a high resolution digital camera. Such a camera is available from Toshiba America Information Systems, Inc. under the model designation CleverDragon. While this image was captured with the CleverDragon camera, a variety of digital cameras will work depending on the application, the necessary resolution, and factors specific to the target area. Factors specific to the target area may include lighting, focal length, size of target area, presence of liquid, sensitivity to a composition, or any other factor likely to affect the image quality or identification of features. Prior to capturing the image, a composition comprising carbon black and titanium oxide was applied to the teeth by aerosol. The captured image shows both the natural features of the target teeth and the black markers of the carbon black. In other embodiments, the markers may be suspended within well known dental agents, like dental bonding agents, dental sealants, or other dental compositions well known in the industry. In some embodiments, the composition comprising the artificial features may be brushed, applied using a foaming liquid or by other means well known in the industry.

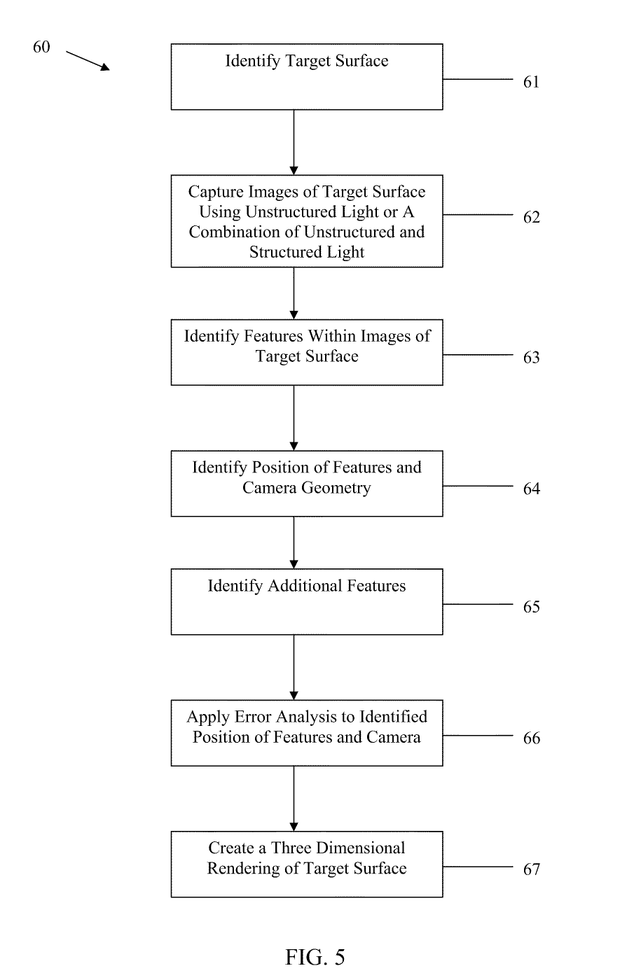

Turning now to FIG. 5 an outline of the steps included in a preferred embodiment or method of the present invention is illustrated generally at 60. First, at step 61 a target surface is identified. The target surface may be intra-oral or extra-oral. Following identification of the target surface, multiple digital images of the target surface are taken at step 62 using unstructured light or a combination of structured and unstructured light by a camera operatively attached to computing system. The images are taken from multiple overlapping positions to allow the capture of specific features within at least two images. Individual features, present in at least two images, are identified at step 63 and the positions determined at step 64. The overlapping images, with identified features, are processed by a general purpose computer (e.g., a personal computer with a Pentium chipset manufactured by Intel) using an algorithm that finds corresponding features of the target area in different images. The corresponding features are determined by analyzing multiple images of the same surface to determine the correspondence for individual features based on error analysis. Once the correspondences for a series of single features on the target area are found, an algorithm identifies additional features at step 65 and repeats the process using error analysis (at step 66) to identify the position of all the identified features. Following identification of features, a three dimensional rendering of the target surface is created at step 67.

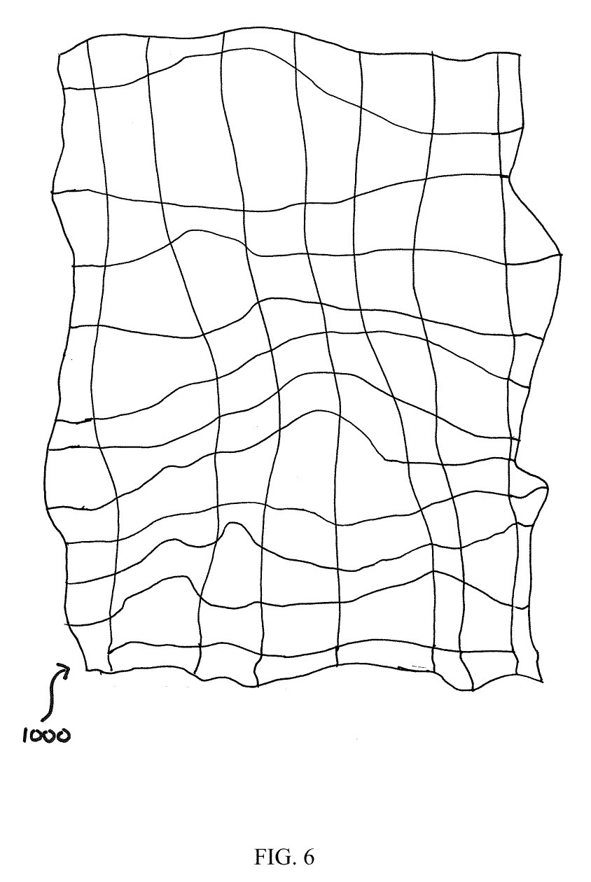

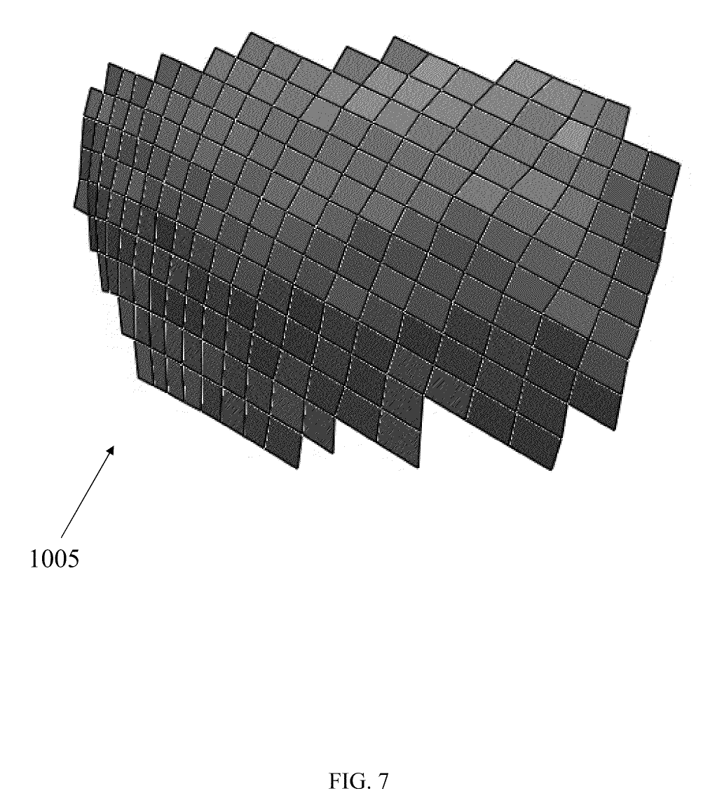

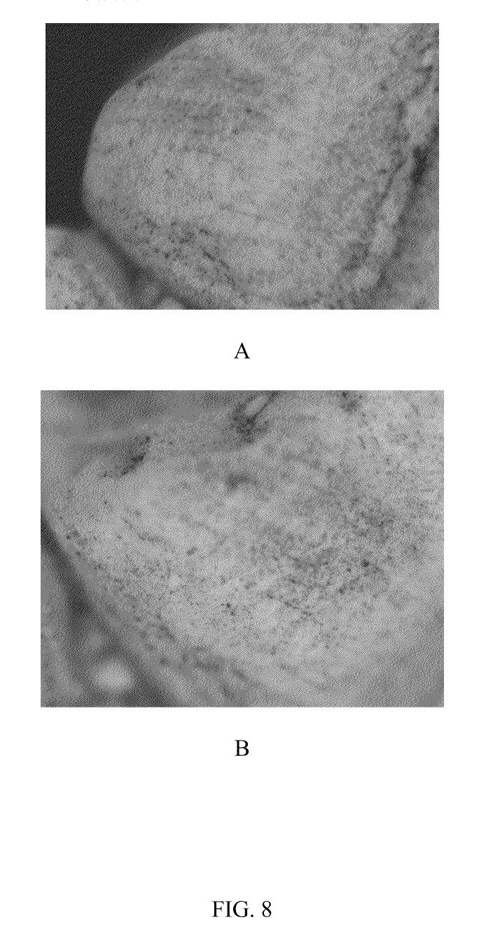

Turning now to FIG. 6 a mesh reconstruction 1000 from working example 2 of a side view of a target tooth. Turning now to FIG. 7 a partial mesh reconstruction 1005 from working example 2 of a side view of a target tooth. FIG. 8 is a first image A of a target tooth from working example 2 and a second overlapping image B of a target tooth from working example 2. Data from these two images was used to construct the partial mesh reconstruction 1005 in FIG. 7.

As used throughout the specification, the term “target area” comprises target teeth, other oral appendages, human appendages, inanimate objects, or generally any surface capable of accepting application of a composition.

As used throughout the specification, the term “target teeth” comprises prepared or unprepared teeth, dental implants, intra-oral landmarks or defect, edentulous ridges or generally any intra-oral feature.

As used throughout this specification, the term “composition” comprises slurries, including granular or dental slurries, gels, aerosols, pastes, liquids, suspensions, or generally any known composition capable of application to a target area.

As used throughout this specification, the term “features(s)” comprises naturally occurring markers on the target area, artificial markers on the target area, granules, particles, markers, darker areas of the target area, lighter areas of the target area, or any other natural or artificial object or data capable of providing pixel data to form correspondences or for other types of image analysis.

It will be appreciated that when discussing image processing within the present invention, the reference to unique or distinct means a feature that can be associated with the corresponding feature in at least a second image. It will be further appreciated that when discussing image processing within the present invention, the reference to granule or marker is one example of a feature, i.e., the granule or marker could be any feature as defined in the present invention. As a further example, a granule may be an individual granule for use as a feature or in the alternative an aggregate of granules may be considered one feature. In addition, a feature may be considered the absence of granules or markers.

### Alternative Embodiment and Environments

While embodiments of the present invention are presented within the context of a dental slurry for intra-oral use, it will be appreciated that the present invention relates more generally to the generation of three dimensional images using unstructured light from multiple two dimensional images.

The present invention relates to both intra-oral and extra-oral applications. Extra-oral applications may include devices, appendages, casts or any other target area capable of accepting a composition. In a certain embodiment, utilization of the present invention determines the surface of a patient's leg for construction of a cast or orthopedic prosthetic. In an embodiment, utilization of the present invention determines the three dimensional surface of patient's foot for construction of an orthotic device.

The present invention relates to both micro and macro-imaging. Micro-imaging may include the use of the present invention for intra-oral applications or other applications requiring the use of a small cameral. Macro-imaging may include the use of the present invention for human appendages, like a leg or an arm, or other application where the target area requires larger image frame and is without size constraints.

It will be appreciated that features will be chosen based on their ability to help identify correspondences between sets of pixels in the various images. Features may be any naturally or artificially occurring marker that may be used in the pixel data to form correspondences or promote any other type of image analysis. In certain embodiments, the features will be used in combination with approximate knowledge of the camera geometry or contextual information, i.e., like a rough idea of the location of the teeth or placement relative to some other contrasting edge, to identify correspondences between sets of pixels in the various images.

More specifically, the features, alone or in combination with contextual information and camera geometry, will identify places within an image where the color appears to change, thereby permitting various known algorithms to search for similar color changes in other images. In preferred circumstances the color changes within the image are rapid, creating a sharp boundary between areas within an image.

It will be appreciated that features of the present invention may be organic or inorganic. For example, some preferred embodiments may comprise organic markets or granules capable of naturally degrading. Organic features may be particularly suitable for use in intra-oral applications. Other embodiments may comprise inorganic features. Inorganic features may be particularly suitable for extra-oral applications based on preferred properties, like the ability to reflect or adsorb light.

The use of compositions with artificial features in the present invention, i.e., markers, granules, and other features not naturally occurring on the target surface, may be chosen based on parameters unique to each application. For example, the texture, location or lighting of the target surface may affect the selection or dispersion of artificial features within the composition. For example, micro-imaging applications, i.e., intra-oral applications, may require smaller artificial features. Macro-imaging applications, i.e., orthotics, may require larger or varying geometry features.

It will be further appreciated that the present invention may find correspondences without granules or markers. For example, the feature may be stripes painted on target area, i.e., target teeth. Like granules or markers, stripes (or similar pattern or elongated markings) may convey shape based on the way the stripes curve and bend, without considering other cues like shading, or prior knowledge of the typical shape. The use of stripes for three dimensional reconstruction is accomplished using the epipolar constraint. For example, when determining correspondences between pixels in two images where the camera positions are known, the epipolar constraint considers that a feature in one image cannot appear just anywhere in the other image. It must appear somewhere along a line in the other image, namely the line formed by considering all possible positions for that feature in the scene, i.e., knowing the position of the feature in one image means that the feature cannot be just anywhere in three dimensional space. The distance from the first camera may be unknown, but everything except that distance is known, and because light travels along a straight line, the feature must lie along a line in three dimensional space, and this line in three dimensional space projects to a line in a second image. Knowing the relative position of two cameras thus reduces the search for correspondences from a two dimensional to a one dimensional problem.

In a preferred embodiment of the present invention, the composition comprises artificial features that fluoresce when subjected to ultraviolet light. In certain embodiments, the uv-illuminating feature is the only artificial feature in the composition. In other embodiments, the uv-illuminating feature is one of at least two artificial features. Some examples of a uv-illuminating features may be a marker or granule coated with uv-illuminating material.

### Algorithm Overview

An embodiment of the present invention allows the user to easily and efficiently generate three dimensional images of target teeth. In an embodiment the target teeth are covered with a dental slurry comprising granules. The target teeth are illuminated by unstructured light and/or ambient light while the user captures multiple overlapping images of the granule covered target teeth using a camera mounted to a wand structure. Each granule may act as a unique or distinct marker on the target teeth. An algorithm calculates a correspondence for each marker using the multiple images and any number of other parameters including a fixed relationship of cameras, fixed pixels, fixed aspect ratio of overlapping images, the optical center of the camera or the coordinate origin. The algorithm uses the calculated correspondences to render three dimensional images of target teeth.

Algorithms and methods for calculating three dimensional images from two dimensional images are well known in the art. For example, a paper by Noah Snavely et al. in the International Journal of Computer Vision entitled Modeling the World from Internet Photo Collections (2007) discusses known techniques and algorithms. The Snavely et al. reference is fully incorporated herein by reference.

As Snavely et al. generally discusses, the first step is finding feature points within each image. Following the identification of feature points, corresponding features are matched between images and a fundamental matrix for the matched features (between images) is established. The geometrically consistent matches between each image pair are organized into a connected set of matching key points across multiple images. Following the identification of correspondences, an image connectivity graph is constructed where each image is a node and an edge exists between any pair of images with matching features. Next, a set of camera parameters (e.g. rotation, translation and focal length) for each image are identified. Following the identification of camera parameters, the information is combined with the identified correspondences and image connectivity graph to compile a three dimensional surface of the target teeth. The image may be refined using the additional images or additional features within the already utilized images.

It will be appreciated that algorithms specific to the present invention will incorporate specific knowledge of the target area. For example, if the target area comprises target teeth, the algorithms may account for the general shape of central incisor as compared to the general shape of first or second molar. The shapes may be stored in memory as a library of various tooth shapes or in other manners. The algorithm may also account for the general difference in size between the teeth of children and adults. Preferred embodiments will account for the information generally known about a target area.

Further, algorithms specific to the present invention will account for the environment of the camera. For example, if the target area comprises target teeth within the intra-oral cavity, the algorithm will account for focus related issues due to the short depth of the field. The algorithm may account for the darkness of the environment, the presence of saliva on the surface of teeth or the difference in reflection of light between bright white and yellow teeth. Preferred embodiments will account for environmental restraints and influences on the camera.

Still further, the algorithm for identifying features will account for the choice of composition applied to the target area. For example, if the composition comprises markers in the shape of spherical granules, the algorithm will account for the properties related to a spherical shape. The algorithm may account for the known properties of a composition comprising markers with single uniform shape and size, or compositions comprising markers with multiple known shapes and sizes. In a preferred embodiment, the algorithm may be adjusted or tailored to the known properties of the features within the composition that is applied to the target area.

In addition to an edge based algorithm technique, the correspondences may also be calculated using a window based algorithm technique or other algorithms known to those skilled in the art.

In an embodiment, the specific technique used may be based on the best error analysis of the available parameters. Therefore, an embodiment may use several different correspondence calculations to render the three dimensional images for a specific set of target teeth.

### Camera

In an embodiment, the digital camera may be designed to be integral with or attached to a rod or wand. Preferably the camera is suitable for intra-oral use. The camera may be pencil like or have a long narrow handle with a flat, wide head. The size and shape of the camera, suitable for intra-oral use, may be standardized or may be provided in different sizes or be adjustable to suit different sized mouths. A light source may be included on the camera, the light source may be located on a second intra-oral device, or the light source may be provided by a device external to the patient's mouth. Additional embodiments may not require a light source as the camera will depend on ambient light. In certain embodiments, the camera utilizes both unstructured light from a provided light source in combination with ambient light. In one embodiment the camera includes an unstructured light source that illuminates both the target teeth having the markers located thereon and the surrounding dentition.

In a preferred embodiment, at least two cameras are attached to a wand like structure at a fixed relationship. The wand like structure has an additional structural feature protruding from the wand and is designed to rest against the teeth. The wand like structure further comprises an unstructured light source. In a preferred embodiment, the wand is inserted into the mouth at preset positions so that the wand structure is positioned at a predetermined distance from the target teeth by resting the protruding structural feature against the gum or target teeth of the mouth. In the preferred embodiment an image is taken from each preset position by each fixed relationship camera while the target teeth are illuminated by the unstructured light source.

Preferably the camera is arranged and configured to be sterilized between uses in differing patients. In one embodiment the camera is capable of withstanding an autoclave. In another embodiment the camera may be inserted into an inexpensive disposable sleeve that permits light to pass through. In yet another embodiment the camera is made of material that may be disinfected using common sterilization cleansers.

The digital camera of the present invention captures images using unstructured light illuminating the granule coated target teeth. The camera can be used to capture specific target teeth, an entire quadrant of the mouth or all four quadrants of the mouth.

### Swishing

In one embodiment, the dental slurry includes granular particles and at least one pharmacologically acceptable or inert carrier. The dental slurry is preferably biodegradable and easily wiped off the target teeth after use—either by manually wiping the teeth or by swishing water or mouth rinse. The dental slurry may vary in viscosity depending on the granules, pharmacological and inert agents or desired property for use. For example, the dental slurry may be viscous enough to allow for painting or daubing the slurry onto target teeth. In another embodiment, the viscosity of the slurry permits swishing the slurry around the intra-oral cavity like a mouthwash. The viscous properties of the slurry may be controlled using a number of safe pharmacological agents including glycerol, polyethylene glycol or other pharmacological agents well known to one of skill in the art.

The dental slurry contains suspended granular particles. The granular particles may have a shape that is spherical, flaky, irregular, rod-like, cone-like, rectangular, or similar geometric shape. The dental slurry may contain a mixture of particle shapes or include one specific particle shape. The spectrum of particle size may include particles spanning several hundred angstroms in one direction or only a couple angstroms. In an embodiment the dental slurry comprises spherical granules between 100 and 200 angstroms. In another embodiment the dental slurry comprises flaky granules ranging from 2 angstroms to 800 angstroms. In still another embodiment, 80% of the granules are spherical and between 50 and 150 angstroms, 10% of the granules are flaky and between 75 and 200 angstroms, and 10% of the granules are rod-like with a size range between 2 and 1000 angstroms.

The granules are designed to temporarily stick to the target teeth and surrounding dentition. When the target teeth are illuminated by unstructured light the granules provide unique or distinct markers within the two dimensional images captured by the camera. These unique or distinct markers are analyzed by an algorithm based on a number of factors including the camera images relationship, the number of pixels, the aspect ratio of the camera, the optical center of the picture and the correspondence for each marker. The algorithm calculates a three dimensional model of the target teeth based on an error analysis of the available parameters.

The composition of the granules within the dental slurry may be determined by the size, features and desired resolution of the target teeth. The composition and size may also be determined by the desired speed of the algorithm and/or number of pixels of the camera. In some embodiments the composition of the granules is suitable for providing distinct markers to small children's teeth. In other embodiments the composition of the granules is suitable for providing distinct markers to large molar or adult teeth. In another embodiment the composition of the granules is suitable for providing distinct markers to teeth when viewed from the buccal, lingual and/or occlusal views.

The dental slurry may contain any number of inert or pharmacologically acceptable agents. For example, viscosity agents, preservatives, antimicrobials and antifungals, water, coloring agents, local anesthetics, or any number of other agents known to one of skill in the art.

A single dental slurry may be employed by swishing around the intra-oral cavity just prior to image capture. In another embodiment, one dental slurry is used for images of specific target teeth and then a second dental slurry is used for images of other target teeth. In another embodiment, a dental slurry is swished around the intra oral cavity and specific target teeth are also marked with a dental slurry using an application brush.

### Apparatus Comprising Granular Particles

It will be further appreciated that an apparatus capable of covering the target teeth may be used in combination with a dental slurry or in place of the dental slurry. In a preferred embodiment, the apparatus is a translucent or transparent mouthpiece that covers the target teeth; however, the apparatus may be any apparatus or material capable of covering the target teeth. Granules may be embedded within or affixed to the apparatus. Upon insertion of the apparatus, i.e., mouthpiece, into a patient's mouth, the granules are visible in combination with the target teeth, thereby permitting visualization of the underlying target teeth in combination with the overlying granules. Thus, a translucent or transparent mouthpiece with embedded granules maybe affixed to the target teeth to replace the use of the dental slurry in the present invention.

In certain embodiments, the translucent or transparent apparatus with embedded granules will be used in conjunction with the dental slurry. For example, images of the target teeth will be taken after application of the dental slurry and then again after insertion of the apparatus, thereby providing an approach to obtaining images with two levels of granules. One will appreciate that the both the dental slurry and the apparatus may have granules of different shapes, size, numbers or properties.

Like the dental slurry, the composition of the granules within the apparatus may be determined by the size, features and desired resolution of the target teeth. The composition and size may also be determined by the desired speed of the algorithm and/or number of pixels of the camera.

In some embodiments the composition of the granules is suitable for providing distinct markers to small children's teeth. In other embodiments the composition of the granules is suitable for providing distinct markers to large molar or adult teeth. In another embodiment the composition of the granules is suitable for providing distinct markers to teeth when viewed from the buccal, lingual and/or occlusal views.

Also similar to the dental slurry, the apparatus may contain any number of inert or pharmacologically acceptable agents. For example, viscosity agents, preservatives, antimicrobials and antifungals, water, coloring agents, local anesthetics, or any number of other agents known to one of skill in the art.

### Operator Software

Preferred user interface software associated with the system is programmed to place the incoming images into a three dimensional view and direct the operator to the portion of target teeth that still requires scanning More specifically, in one embodiment the target teeth are selected by the operator on the computer system. The computer system then directs the operator to scan the target teeth at predetermined positions resulting in overlapping images that provide the algorithm with the data necessary to construct an entire three dimensional image of the target teeth. It will be appreciated, that the predetermined positions may not be exact based on possible operator error. In another embodiment the camera is preprogrammed with a predetermined number of positions from which the camera captures images. The predetermined positions provide overlapping coverage of the entire region including the target teeth. In another embodiment the regions of target teeth are marked on a three dimensional model shown on the display and allow the operator of the camera to determine what regions of the target teeth remain to be imaged.

The video display unit may be used to show the captured images both prior to and after algorithmic analysis. Prior to algorithmic analysis the raw images are comprised of individual pixels that represent the different target teeth. After algorithmic analysis, the individual pixels are used to construct a three dimensional rendering of the target teeth including an accurate portrayal of the features associated with the target teeth.

In a preferred embodiment the display communicates to the operator the accuracy of each rendered section of target teeth, thereby allowing the operator to increase accuracy of specific target regions by acquiring more images of that region.

In an embodiment, the display may show both the pixel and three dimensional rendering simultaneously. In another embodiment the three dimensional rendering of the target teeth is displayed in near real time with the scanning of the target teeth. In another embodiment the display provides directions to the operator in the form of word, color, sound or similar commands. In an embodiment, the directions are based on the incoming images associated with the imaged target teeth.

In an embodiment the display has several input devices including a keyboard and the camera and the operator can perform varying tasks simultaneously, including imaging the target teeth.

### Working Example 1

In a preferred embodiment a patient swishes a granular slurry around their mouth before spitting the remaining slurry out. Upon completion of the swishing, the patient's target teeth are temporarily covered with granular particles. The operator then inserts a wand structure into the patient's mouth. The wand structure comprises at least one intra-oral camera and at least one source of unstructured light. In addition the wand structure comprises a fixed inert protruding structure. The fixed inert protruding structure protrudes from the wand structure farther than either the camera or the unstructured light source.

The operator starts at a predetermined location within patient's mouth based on the operator software. At each predetermined location, the operator presses the wand structure toward the gum of the target teeth until the fixed inert protruding structure contacts the gum. At the point of contact, the operator captures multiple images from the camera(s) at each predetermined location (e.g., by rotating the wand about the point of contact). The protruding structure allows the operator to capture each series of images at a predetermined distance from the target teeth.

As the operator continues to progress through the series of fixed locations, the user software begins calculating the incoming data using an algorithm. The algorithm utilizes the fixed relationship between the camera(s) and the fixed pixel and aspect ratio resulting from the protruding structure to calculate a correspondence for each unique or distinct marker identified in overlapping images.

The algorithm uses these calculated correspondences to display a generated three dimensional image of the target teeth on the operator interface. The operator interface allows the operator to track the progression and accuracy of three dimensional images resulting from the target teeth.

Those skilled in the art will appreciate that the present invention may be embodied by forms that are not disclosed without departing from the spirit or fundamental attributes thereof. While the description of the present invention discloses only some embodiments, a skilled artisan will appreciated that other variations are contemplated as being with the scope of the present invention. Accordingly, the present invention is not limited in the particular embodiments which have been described in detail therein. Since many embodiments of the invention can be made without departing from the spirit and scope of the invention, the invention resides in the claims hereinafter appended.

### Working Example 2

Target teeth, i.e., a cast of teeth from a patient's mouth, were covered with a dental slurry comprising carbon black and titanium dioxide. The slurry was sprayed on as an aerosol. Three overlapping images were captured of the target teeth covered with dental slurry using unstructured light and a Toshiba CleverDragon camera connected to a frame grabber. Following image capture, a small robust set of features were selected from the three images. The robust set of features included twenty unique features common to at least two images and comprised both artificial features from the dental slurry and naturally occurring features on the target teeth.

Using these twenty unique features, the camera geometry and position of the twenty features were identified based on algorithms common in the industry, i.e., a pin hole camera model using minimized reprojection error. Following identification of the camera geometry and position of the twenty features using minimized reprojection error, an additional, much larger set of second features were chosen on the visible surface of one of the teeth. The second set of features comprised thousands of small rectangular pixel areas in the second image on the visible surface of a target tooth. Each of these features was matched with a corresponding area in the first image, using hierarchical refinement and epipolar and continuity constraints.

The original position of the twenty features and camera geometry were refined, and the positions of the newly identified features in space were obtained, both by minimizing the reprojection error. Following this refinement, the points corresponding to the second set of features were connected in a quadrilateral mesh (FIG. 6). This mesh was then used for a three dimensional rendering of the target tooth.

What is claimed is:

1. A dental slurry for creating an electronic three dimensional model of target teeth using unstructured light, comprising:
at least one inert or pharmacologically acceptable carrier; and
granular particles suspended in the carrier.

2. A method for creating electronic three dimensional images of target teeth using unstructured light comprising:
swishing a dental slurry having particles suspended therein within the mouth of a patient;
acquiring overlapping images of target teeth covered by the particles;
combining the images into a three dimensional electronic model using an algorithm that finds correspondences for distinct particles based on error analysis; and
displaying the three dimensional electronic model.

3. A kit for producing three dimensional images of target teeth comprising:
a container, wherein the container holds granular particles and an inert or pharmacologically acceptable carrier constituting a dental slurry.

4. The kit of claim 3, further comprising additional containers.

5. The kit of claim 4, wherein an additional container holds dental slurry comprising granular particles of different size distribution.

6. A system for producing three dimensional images of target teeth comprising:
a dental slurry, wherein the dental slurry comprises granular particles and inert or pharmacologically acceptable agent;
a digital camera;
a source of unstructured light;
a computing system; and
a display.

7. A method for creating electronic three dimensional images of facio-cranial targets using unstructured light comprising:
applying a slurry having particles suspended therein onto the facio-cranial target;
acquiring overlapping images of the facio-cranial target covered by the particles;
combining the images into a three dimensional electronic model using an algorithm that finds correspondences for distinct particles based on error analysis; and
displaying the three dimensional electronic model.

8. The method of claim 7, wherein the facio-cranial target is a dental device.

9. The method of claim 8, wherein the dental device is a study cast.

10. The method of claim 9, wherein the slurry is applied to the study cast by spraying or dipping.

11. The method of claim 8, wherein the dental device is a study cast, a dental prosthesis, a dental implant, a dental appliance or a bite registration wafer.
